(19) United States
(12) Patent Application Publication
(10) Pub. No.: US 2004/0215109 A1
(43) Pub. Date: Oct. 28, 2004

(54) Helical guidewire

(76) Inventors: Edward D. Pingleton, Laguna Niguel, CA (US); Nabil Hilal, Laguna Niguel, CA (US); Said S. Hilal, Coto De Caza, CA (US)

Correspondence Address: APPLIED MEDICAL RESOUCES CORPORATION, 22872 Avenida Empresa, Rancho Santa Margarita, CA, 92688, US

(21) Appl. No.: 10/829,343

(22) Filed: Apr. 21, 2004

Related U.S. Application Data
(60) Provisional application No. 60/464,878, filed on Apr. 23, 2003.

(51) Int. Cl. A61B 5/00

(52) U.S. Cl. 600/585

(57) ABSTRACT

A guidewire is formed from a wire twisted around a portion of a longitudinal axis. The guidewire has a first longitudinal edge and a second longitudinal edge. The guidewire may be formed from a kink-resistant material such as Nitinol, stainless steel or titanium. The first and second longitudinal edges form a helical configuration such that the guidewire is capable of transmitting torque while remaining flexible. In one embodiment of the invention, the first and second longitudinal edges and the amount of twisting or pitching remain constant throughout the full length of the guidewire. In other embodiments of the invention, the dimensions of the longitudinal edges and the amount of twisting or pitching may vary widely over the full length or different sections of the guidewire depending on the intended application of the wire. The guidewire may be formed from a single twisted wire of different cross-sections or a plurality of wires having similar or different shapes twisted together around a longitudinal axis. The plurality of wires may be two wires having generally rectangular cross-sections with different dimensions, one wire having a generally rectangular cross-section and the other wire having a generally round cross-section, one wire having a generally rectangular cross-section and the other wire having a generally triangular cross-section, and a plurality of wires having generally round cross-sections, and the wires may be helically twisted together forming a guidewire. The wires may be held together by the sheer force of being twisted together around a longitudinal axis, or the wires may be glued or soldered together, either along the entire length of the wires or by spot gluing or soldering.

This is a non-provisional application claiming the priority of provisional application Ser. No. 60/464,878, filed on Apr. 23, 2003, entitled “Helical Guidewire,” which is fully incorporated herein by reference.

### BACKGROUND OF THE INVENTION

1. Field of the Invention

This invention generally relates to surgical guidewires and, more specifically, to a helically twisted wire providing flexibility, kink-resistance and stiffness.

2. Discussion of the Prior Art

Surgical guidewires are well known for placing and guiding catheters and other devices in lumens of the human body. In particular, the surgical guidewires are primarily used to facilitate the placement of catheters and endoscopic instruments within the tortuous paths of body conduits. For example, if it is desirable to place a catheter within the vascular system of a human body, a guidewire is first inserted into the vessel and guided through the tortuous path desired for the catheter, the catheter is then threaded over the guidewire. As the catheter is inserted and advanced over the placed guidewire, it tends to follow the direction of the guidewire so that it ultimately negotiates the same tortuous path. Once the catheter is in its final operative position, the guidewire can be removed leaving the catheter to perform its desired therapeutic function. Guidewires are currently used in, among other fields, cardiology, urology, radiology, electrophysiology and gastroenterology.

The performance of a guidewire is influenced by certain characteristics such as flexibility and stiffness. The flexibility or steerability of a guidewire is important especially when a tortuous path must be navigated to reach the target site as is commonly encountered when placing a catheter, such as an angioplasty catheter, in the coronary arteries. Steering is executed from the proximal end of the guidewire by rotating, pushing and pulling on the guidewire to cause corresponding movement at the distal tip of the wire. The distal tip may be bent so that when rotated the tip can be directed toward a selected one of several vascular branches. Another important characteristic of guidewires is its stiffness. That is, if a guidewire is not stiff enough, it may kink during use. Kinking is a sharp deformation or bending that may result from attempting to pass the guidewire through a relatively hard, calcified lesion, a mostly occluded vessel section or a very tortuous vascular section. Once kinked, the guidewire loses its controllability and usually must be discarded. Consequently, the procedure may have to be aborted and a new guidewire selected, reinserted and again manipulated and advanced to the target site.

As such, construction of guidewires typically involves a balance between flexibility and stiffness. In particular, it is important that the guidewire be sufficiently flexible so that it does not damage the wall of the blood vessel into which it is being inserted and to adapt itself to the path of the blood vessel. At the same time, a stiff guidewire is preferred since it transmits torque along the whole body and is therefore more pushable. A stiff guidewire is also less likely to deform or kink during use. Many attempts have been made to improve the flexibility and stiffness of surgical guidewires.

In the past, for example, guidewires have been formed with solid cores or shafts which extend along substantially the entire length of the guidewires from its proximal end to its distal end. In order to increase the flexibility at the distal end of the guidewire, the core or shaft has been tapered and springs have been formed on the tapered surface. This construction has provided guidewires with distal tips having increased flexibility. However, the core still extends along the entire length of the guidewire with this construction so that even in the region where flexibility is desired such as the distal tip, the core has tended to add stiffness. Solid cores have also presented a problem when the guidewire has been severely bent. In a typical construction where the core extends throughout the length of the guidewire, a severe bend has forced the core to yield making it difficult for the distal tip to recover to its original configuration. As such, although improvements in guidewire construction have been made, guidewire construction still involves trade-offs and compromises between flexibility and stiffness, and there is still a need in the art for an improved guidewire providing these competing characteristics.

### SUMMARY OF THE INVENTION

This invention is directed to a flexible yet stiff guidewire formed from a wire twisted around at least a portion of a longitudinal axis. The guidewire has a first longitudinal edge and a second longitudinal edge. The guidewire may be formed from a kink-resistant material such as Nitinol, stainless steel, titanium or other metallic or polymer alloy having spring wire characteristics. The first and second longitudinal edges form a helical configuration such that the guidewire is capable of transmitting torque while remaining flexible. In particular, the helical configuration of the guidewire provides a stiff structure capable of transmitting torque along the length of the wire while enhancing pushability of the wire. At the same time, the helical configuration provides lower friction and less drag force than conventional guidewires. Furthermore, the helical configuration is sufficiently flexible so that it does not damage the body conduit such as the wall of a blood vessel into which it is being inserted and to adapt itself to the path of the body conduit. In one embodiment of the invention, the first and second longitudinal edges and the amount of twisting or pitching remain constant throughout the full length of the guidewire. It is appreciated that the dimensions of the longitudinal edges and the amount of twisting or pitching may vary over the full length of the wire or different sections of the wire depending on the intended application of the guidewire. The diameter of the guidewire may vary from about 0.010″ to about 0.060″, and the length of the guidewire may vary from about 100 cm to about 200 cm depending on the application of the wire.

The guidewire may be formed from a single twisted wire or a plurality of wires having similar or different shapes twisted together around a longitudinal axis. In one embodiment of the invention, two wires may have generally rectangular cross-sections with different dimensions and may be helically twisted together forming a flexible, kink-resistant and stiff guidewire. In this embodiment, the two rectangular-shaped wires may be held together by the sheer force of being twisted together around the longitudinal axis. The wires may also be glued or soldered together, either along the entire length of the wires or by spot gluing or soldering. Furthermore, the two wires may be coextruded or coated with plastic. In another embodiment of the invention, one wire may have a generally rectangular cross-section and the other wire may have a generally round cross-section and the two wires may be helically twisted together forming a flexible, kink-resistant and stiff guidewire. In yet another embodiment of the invention, one wire may have a generally rectangular cross-section and the other wire may have a generally triangular cross-section and the two wires may be helically twisted together forming a flexible, kink-resistant and stiff guidewire. In yet another embodiment of the invention, a plurality of wires may have generally round cross-sections and may be helically twisted together forming a flexible, kink-resistant and stiff guidewire.

It is appreciated that each of the twisted wire may have different cross-sections including but not limited to round, oval, square, triangular, rectangular, pentagonal, hexagonal and any multisided shape.

These and other features of the invention will become more apparent with a discussion of the various embodiments in reference to the associated drawings.

### DESCRIPTION OF THE DRAWINGS

FIG. 1 illustrates a full-length helical wire having a generally rectangular cross-section in accordance with a first embodiment of the invention;

FIG. 2 illustrates a cross-sectional view of a helical wire having a rectangular shape;

FIGS. 3 and 5 illustrate additional embodiments of helical wires having generally rectangular cross-sections with varying longitudinal edges dimensions;

FIG. 4 illustrates a helical wire having a generally rectangular cross-section and varying amount of twisting or pitching over the entire length of the wire in accordance with another embodiment of the invention;

FIG. 6 illustrates a cross-sectional view of two wires having generally rectangular cross-sections with different dimensions helically twisted together in accordance with another embodiment of the invention;

FIG. 7 illustrates two wires having generally rectangular cross-sections with different dimensions helically twisted together forming a guidewire in accordance with the embodiment of FIG. 6;

FIG. 8 illustrates a cross-sectional view of two wires, one having a generally rectangular cross-section and one having a generally round cross-section, helically twisted together in accordance with another embodiment of the invention;

FIG. 9 illustrates two wires, one having a generally rectangular cross-section and one having a generally round cross-section, helically twisted together forming a guidewire in accordance with the embodiment of FIG. 8;

FIGS. 10 and 11 illustrate two wires, one having a generally rectangular cross-section and one having a generally triangular cross-section, helically twisted together forming a guidewire in accordance with another embodiment of the invention;

FIGS. 12 and 13 illustrate a plurality of wires having generally round cross-sections helically twisted together forming a guidewire in accordance with another embodiment of the invention;

FIGS. 14-16 illustrate additional embodiments of helical wires having generally square cross-sections with varying amount of twisting or pitching over the entire length of the wires; and

FIG. 17 illustrates a guidewire of the invention coated with plastic to provide additional rigidity and/or torquability.

### DESCRIPTION OF AND BEST MODE OF THE INVENTION

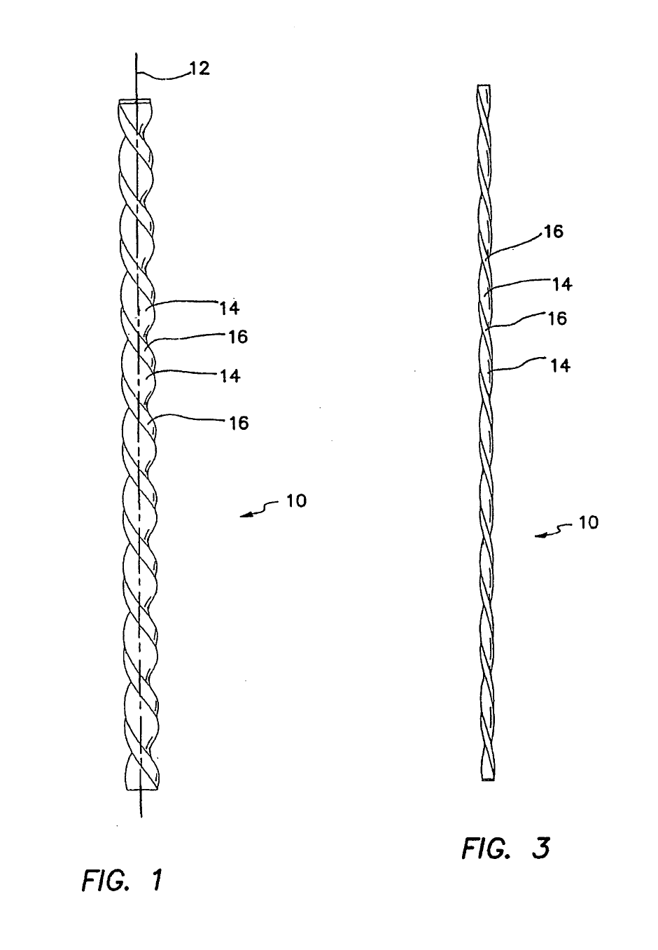

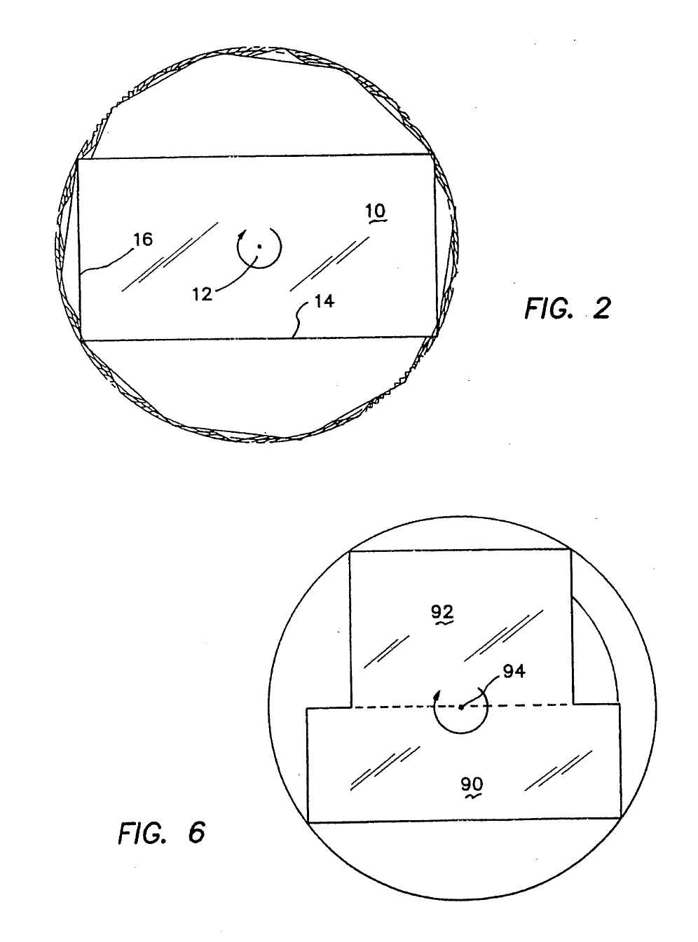

FIG. 1 illustrates a helical guidewire 10 in accordance with the first embodiment of the invention. Guidewire 10 is formed of a biocompatible material twisted around a longitudinal axis 12. FIG. 2 illustrates a cross-section of the guidewire 10 having a first longitudinal edge 14 and a second longitudinal edge 16. Guidewire 10 is preferably formed from a kink-resistant material such as Nitinol, stainless steel, titanium or other metallic or polymer alloy having spring wire characteristics. In general, although guidewire 10 can be formed from other materials, metals are desirable as they enhance fluoroscopic imaging. Metals further enhance torquability and pushability along the entire guidewire, both at the proximal portion and at the distal portion.

The guidewire 10 may be formed by rotating a piece of wire about its longitudinal axis 12 or by molding and/or grinding. The first and second longitudinal edges 14 and 16, respectively, thus take a helical configuration such that the guidewire 10 is capable of transmitting torque while remaining flexible. Stated another way, a feature of the invention is the resulting twisted wire provides relatively uniform flexibility and pushability. Moreover, the construction of the invention does not need a tip or spring typically required in the prior art to provide pushability and torque-transmitting characteristics desirable in a guidewire. The guidewire 10 has a twisting diameter that may vary from about 0.010″ to about 0.060″, and the length of the guidewire 10 may vary from about 100 cm to about 200 cm depending on the application of the wire. It is understood by one skilled in the art that the dimensions of a wire depend on the application of the wire.

To use the guidewire for insertion, the guidewire is normally threaded to a desired position. As stated above, if it is desirable to place a catheter within the vascular system of a human body, a guidewire is first inserted into the vessel and guided through the tortuous path desired for the catheter, the catheter is then threaded over the guidewire. As the catheter is inserted and advanced over the placed guidewire, it will tend to follow the direction of the guidewire so that it ultimately negotiates the same tortuous path. Once the catheter is in its final operative position, the guidewire can be removed leaving the catheter to perform its desired therapeutic function. It should be noted that the guidewire of the invention could be in used in, among other fields, cardiology, urology, radiology, electrophysiology and gastroenterology.

In one embodiment of the invention as illustrated in FIGS. 1 and 2, the first and second longitudinal edges 14 and 16 of the guidewire 10 and the amount of twisting or pitching remain constant throughout the full length of the guidewire 10. As explained above, the helical configuration of the guidewire 10 provides a stiff structure capable of transmitting torque along the full length of the wire while enhancing pushability of the entire wire. At the same time, the helical configuration provides lower friction and less drag force than conventional guidewires. Moreover, the helical configuration is sufficiently flexible so that the guidewire 10 does not damage the body conduit such as the wall of a blood vessel into which it is being inserted and to adapt itself to the path of the body conduit. It is appreciated, however, that the dimensions of the longitudinal edges and the amount of twisting or pitching may vary widely over the full length or different sections of the guidewire depending on the intended application of the wire as further described below in other exemplary embodiments of the invention. It is further appreciated that the single twisted wire may have different cross-sections including but not limited to round, oval, square, triangular, rectangular, pentagonal, hexagonal and any multisided shape.

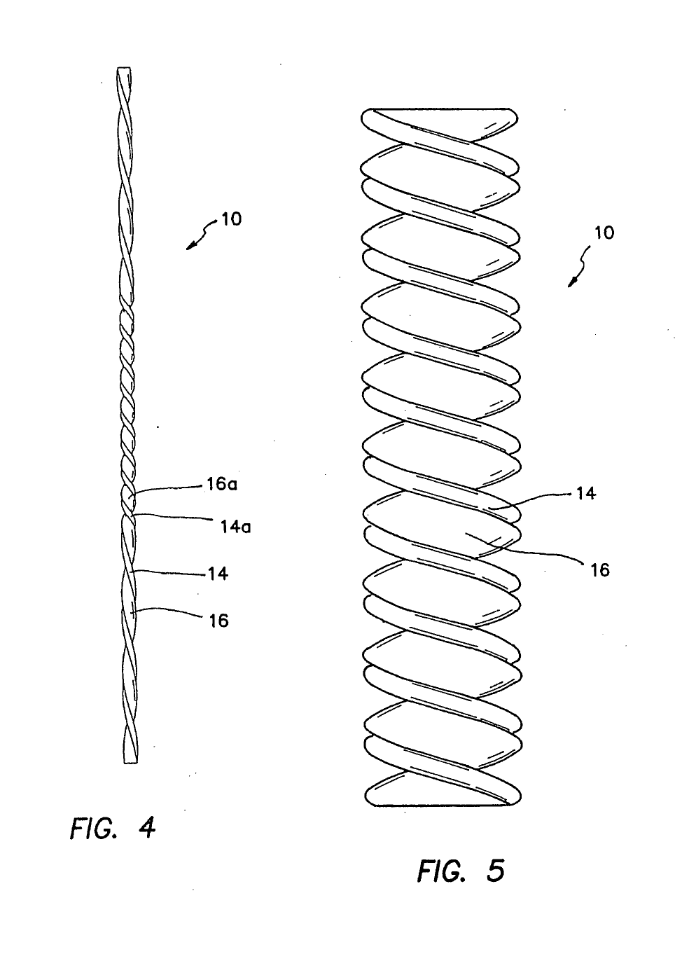

Referring to FIGS. 3-5, additional embodiments of helically twisted wires having generally rectangular cross-sections with varying longitudinal edges dimensions and varying amount of twisting or pitching over the full length of the wires are illustrated. It is appreciated that the size or diameter of the body conduit or lumen into which the guidewire is being inserted is an important factor in determining the dimensions of the wire, and the amount of twisting or pitching of the wire may vary wire stiffness.

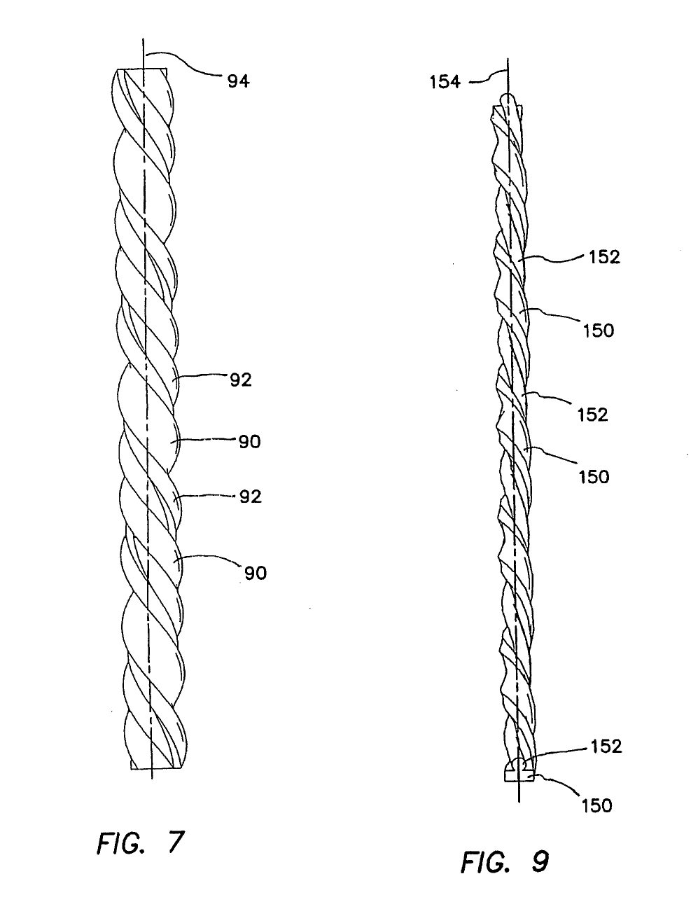

Referring to FIGS. 6 and 7, there are shown two wires having generally rectangular cross-sections with different dimensions helically twisted together forming a guidewire in accordance with another embodiment of the invention. In this embodiment, two rectangular-shaped wires 90 and 92 may be held together by the sheer force of being twisted together around longitudinal axis 94. The wires 90 and 92 may also be glued or soldered together, either along the entire length of the wires or by spot gluing or soldering. Furthermore, the two wires may be coextruded or coated with plastic as further described below.

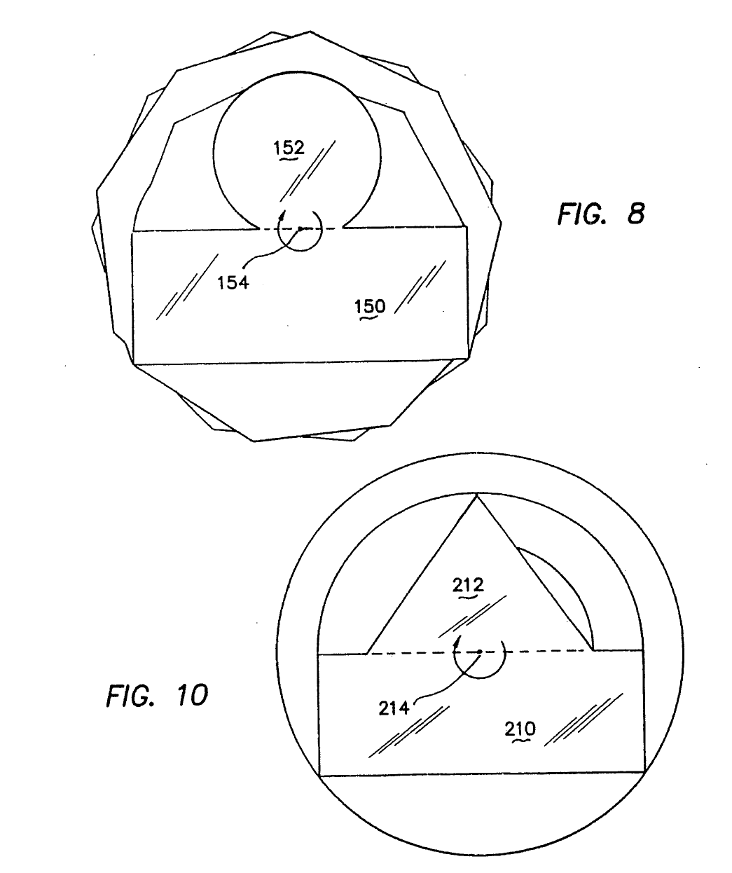

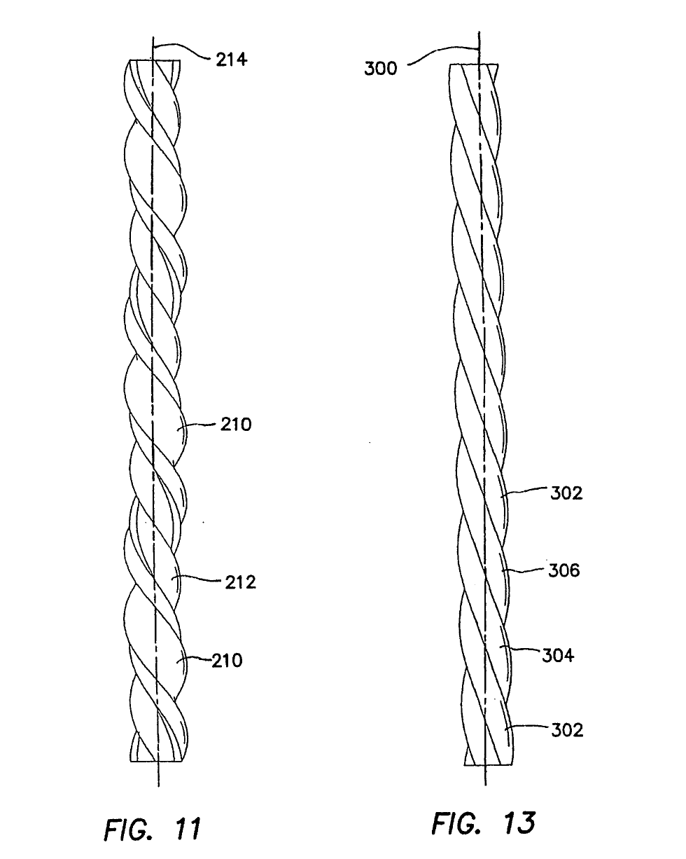

FIGS. 8 and 9 illustrate another embodiment of the invention where two wires, one having a generally rectangular cross-section 150 and one having a generally round cross-section 152, are helically twisted together forming a flexible, kink-resistant and stiff guidewire. As illustrated in FIG. 8, rectangular-shaped wire 150 and round-shaped wire 152 may be held together by being twisted together around longitudinal axis 154, or they may be glued or soldered together. FIGS. 10 and 11 illustrate another embodiment of the invention where two wires, one having a generally rectangular cross-section 210 and one having a generally triangular cross-section 212, are helically twisted together forming a flexible, kink-resistant and stiff guidewire. Similar to the above described embodiments of the invention, rectangular-shaped wire 210 and triangular-shaped wire 212 may be held together by being twisted together around longitudinal axis 214, or they may be glued or soldered together.

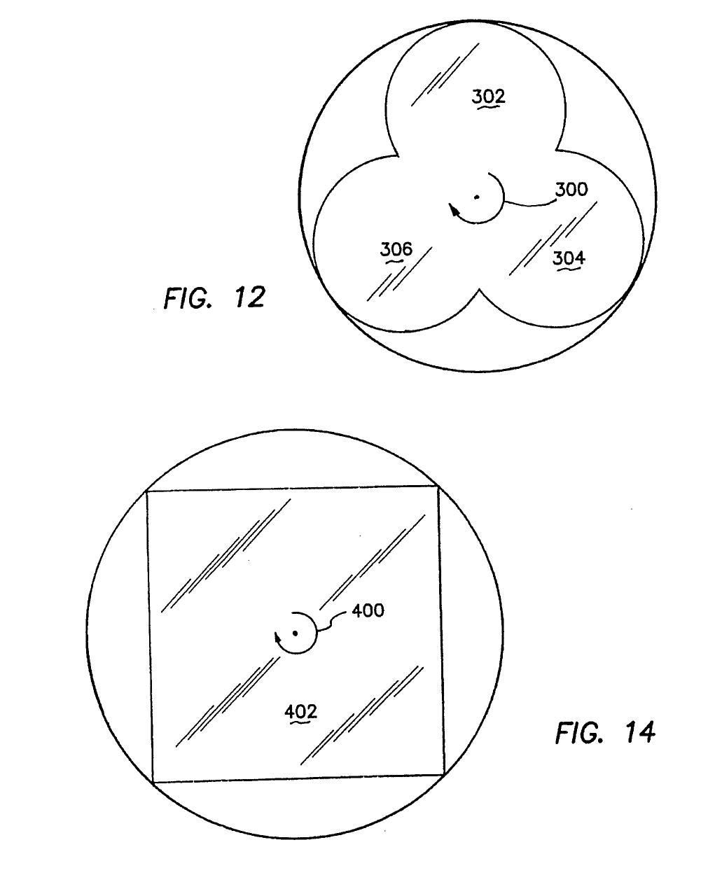

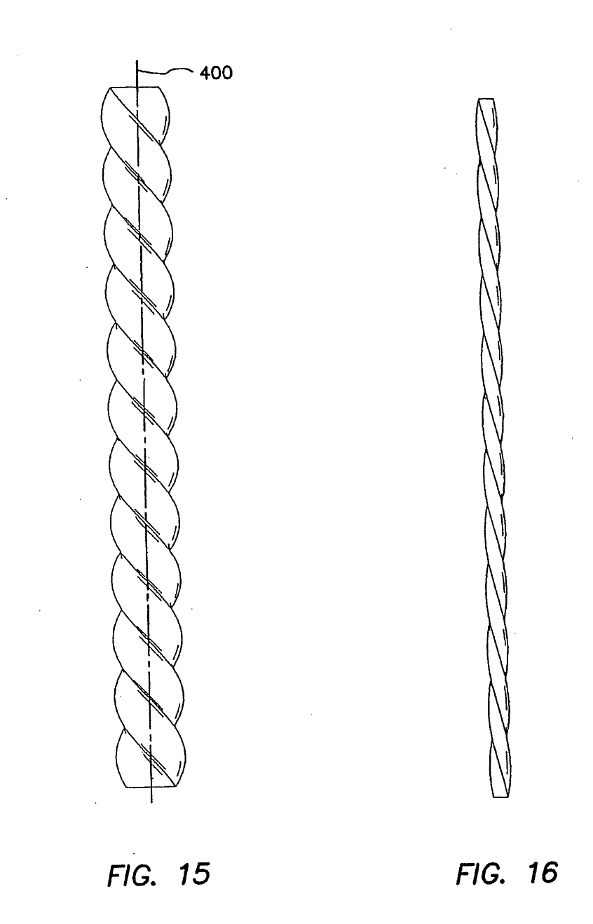

In another embodiment of the invention, FIGS. 12 and 13 illustrate a plurality of wires having generally round cross-sections helically twisted together forming a flexible, kink-resistant and stiff guidewire. In yet additional embodiments of the invention, FIGS. 14-16 illustrate helical wires having generally square cross-sections with varying amount of twisting or pitching over the full length of the wires.

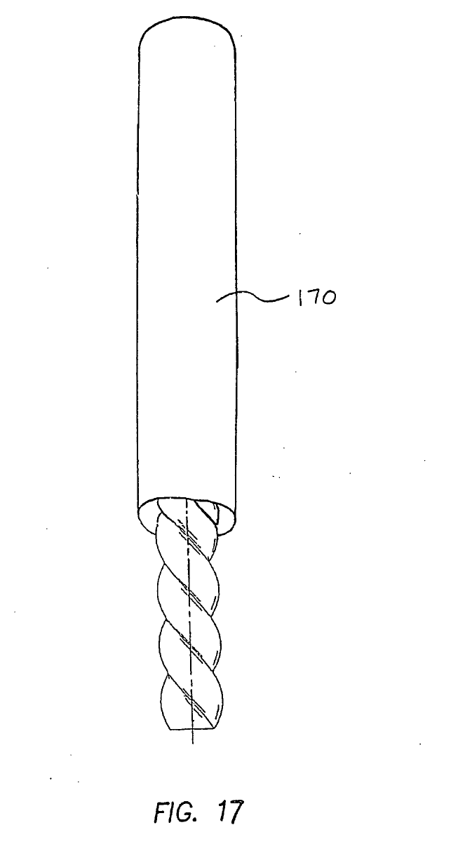

It is appreciated that in each of the above embodiments of the invention, any portion of the wire such as the tip of the wire may be grinded or tapered before twisting to further enhance flexibility or steerability. It is further appreciated that the twisted guidewire may be attached to a shaft, which may be a solid core wire or a hollow tube. It is further appreciated that a tip spring may be mounted at the distal end of the guidewire. It is further appreciated that in each of the above embodiments, the guidewire may be coextruded, coated or encased in a plastic sheath 170 over the entire guidewire or any portion thereof to provide additional rigidity and/or torquability as illustrated, for example, in FIG. 17. The sheath 170 may be formed from a polyethylene material and may have a lubricous coating. Different materials and surface treatments may also be considered for the various sheaths.

Accordingly, the helically twisted guidewires of the invention greatly improve flexibility, kink-resistance and stiffness over the prior art designs, which only address some of these characteristics by adjusting either the shaft or core of the wire or the tip of the wire. In contrast, the designs of the invention address all these characteristics by rotating or twisting the full length of the wire, which may be a single wire or a combination of differently shaped wires, from the proximal end of the core to the distal end of the tip.

Although exemplary embodiments of the invention have been shown and described, many other changes, modifications and substitutions will now be apparent to those of ordinary skill in the art, without necessarily departing from the spirit and scope of this invention.

1. A surgical guidewire, comprising:
a wire having a length extending along a longitudinal axis and a generally rectangular cross-section having a first longitudinal edge and a second longitudinal edge, the wire twisting helically around the longitudinal axis for at least a section of the length of the wire.

2. The surgical guidewire of claim 1, wherein the wire is formed from a kink-resistant material including Nitinol, stainless steel, titanium or other metallic or polymer alloy having spring wire characteristics.

3. The surgical guidewire of claim 1, wherein the helical configuration of the wire enhances at least one of torquability and pushability of the wire.

4. The surgical guidewire of claim 1, wherein the amount of twisting or pitching of the wire varies over at least a portion of the wire.

5. The surgical guidewire of claim 1, wherein the amount of twisting or pitching of the wire remains constant over the entire length of the wire.

6. The surgical guidewire of claim 1, wherein the dimensions of the first and the second longitudinal edges vary over the entire length of the wire or over different sections of the wire.

7. The surgical guidewire of claim 1, wherein the twisted wire has a diameter varying from about 0.010″ to about 0.060″.

8. The surgical guidewire of claim 1, wherein the length of the wire varies from about 100 cm to about 200 cm.

9. The surgical guidewire of claim 1, further comprising at least another wire helically twisting around or together with the first wire.

10. The surgical guidewire of claim 9, wherein the at least another wire has a cross-section having a round, an oval, a square, a triangular, a rectangular, a pentagonal, a hexagonal or any multisided shape.

11. The surgical guidewire of claim 1, further comprising a second wire helically twisting together with the first wire around the longitudinal axis.

12. The surgical guidewire of claim 11, wherein the second wire has a generally rectangular cross-section having a first longitudinal edge and a second longitudinal edge different from the first longitudinal edge and the second longitudinal edge of the first wire.

13. The surgical guidewire of claim 11, wherein the second wire has a generally round cross-section.

14. The surgical guidewire of claim 11, wherein the second wire has a generally triangular cross-section.

15. The surgical guidewire of claim 11, wherein the first and the second wires are held together by the sheer force of being twisted together around the longitudinal axis.

16. The surgical guidewire of claim 11, wherein the first and the second wires are affixed or attached together.

17. The surgical guidewire of claim 11, wherein the first and the second wires are affixed or attached together by spot gluing or soldering.

18. The surgical guidewire of claim 11, further comprising a coating over the entire guidewire or over a portion of the first and the second wires.

19. The surgical guidewire of claim 11, wherein the entire guidewire or a portion of the first and the second wires is coextruded with plastic.

20. The surgical guidewire of claim 1, wherein the wire further comprises a tip.

21. The surgical guidewire of claim 20, wherein the tip is grinded or tapered before twisting to further enhance flexibility or steerability.

22. The surgical guidewire of claim 1, further comprising a tip spring mounted at a distal end of the wire.

23. A surgical guidewire, comprising:
a first wire having a first cross-section and extending along a longitudinal axis; and
a second wire having a second cross-section extending along the longitudinal axis,
wherein the first wire and the second wire are helically twisted together around at least a section of the longitudinal axis.

24. The surgical guidewire of claim 23, wherein the helical configuration of the first and the second wires enhances torquability while maintaining flexibility.

25. The surgical guidewire of claim 23, wherein at least one of the first and the second wires is a kink-resistant wire formed from Nitinol, stainless steel, titanium or other metallic or polymer alloy having spring wire characteristics.

26. The surgical guidewire of claim 23, wherein the first and the second cross-sections of the wires are generally round.

27. The surgical guidewire of claim 23, wherein the first and the second cross-sections of the wires are generally similar in at least shape and size.

28. The surgical guidewire of claim 23, wherein the first and the second cross-sections of the wires are generally different in at least shape and size.

29. The surgical guidewire of claim 27 or claim 28, wherein the shape includes any geometric shape including circular, oval, square, triangular, rectangular, pentagonal, hexagonal or any multisided shape.

30. The surgical guidewire of claim 23, further comprising at least a third wire helically twisting around or together with the first and the second wires.

31. A surgical guidewire, comprising:
a first wire having a first cross-section and extending along a longitudinal axis; and
a second wire having a second cross-section,
wherein the second wire is twisted around at least a section of the first wire.

32. The surgical guidewire of claim 31, wherein at least one of the first and the second wires is a kink-resistant wire formed from Nitinol, stainless steel, titanium or other metallic or polymer alloy having spring wire characteristics.

33. The surgical guidewire of claim 31, wherein the first and the second cross-sections of the wires are generally similar in at least shape and size.

34. The surgical guidewire of claim 31, wherein the first and the second cross-sections of the wires are generally different in at least shape and size.

35. The surgical guidewire of claim 33 or claim 34, wherein the shape includes any geometric shape including circular, oval, square, triangular, rectangular, pentagonal, hexagonal or any multisided shape.
